(19) United States
(12) Patent Application Publication
(10) Pub. No.: US 2009/0205991 A1
(43) Pub. Date: Aug. 20, 2009

(54) MEDICAL TUBING BAG

(76) Inventor: Robert J. Lill, Wichita, KS (US)

Correspondence Address: LATHROP & GAGE LLP, 2345 GRAND Boulevard, SUITE 2400, KANSAS CITY, MO, 64108, US

(21) Appl. No.: 12/399,845

(22) Filed: Mar. 6, 2009

Related U.S. Application Data
(63) Continuation-in-part of application No. 11/586,941, filed on Oct. 26, 2006.
(60) Provisional application No. 61/034,415, filed on Mar. 6, 2008.

(51) Int. Cl. A61B 19/02

(52) U.S. Cl. 206/438

(57) ABSTRACT

A medical tubing bag disclosed herein includes an interior tube receiving area, and first and second openings. In one embodiment, a first guide is adjacent the first opening. The bag is used, in embodiments, to handle surplus tubing between a concentrator and a cannula. Depending on the embodiment, the tubing can be retracted and released from the bag either manually or using some sort of reel system.

### RELATED APPLICATIONS

This Application claims priority to U.S. Provisional Patent Application Ser. No. 61/034,415 filed Mar. 6, 2008, and is a Continuation-In-Part of application Ser. No. 11/586,941, filed Oct. 26, 2006. Both of the above-mentioned applications are incorporated herein by reference.

### BACKGROUND

Medical tubing, including oxygen tubing and other types of tubing, is commonly used to connect a user to a medical device (e.g., an oxygen source). When a user is moving or being moved in relation to the medical device, there is a strong tendency for the tubing to become kinked, unconnected from the medical device, and/or heaped or strewn in inconvenient and potentially dangerous manners.

Various devices have been created to increase the mobility of people using medical tubing. Of these, many provide ways for coupling the medical tubing and the medical device to the user, such as those disclosed in U.S. Pat. Nos. 4,383,528; 4,438,764; 4,739,913; 5,370,113; 5,676,135; and 6,003,744 and in U.S. Pat. Publication No. 2002/0104860. While these may be useful in situations where the user is moving over large areas, they are not ideal for users moving over smaller areas because the user is having to carry or otherwise maneuver the weight of the medical device unnecessarily. Previously, there has not been a convenient way for the user to travel within a defined radius of a generally-stationary medical device without either: 1) enduring problems (such as those mentioned above) with the medical tubing that connects the user to the medical device; or 2) exerting excessive attention and energy to maintain the medical tubing in an orderly fashion.

### SUMMARY

A medical tubing bag that organizes medical tubing and allows a user to easily move within a defined radius of a generally-stationary medical device would increase mobility and user safety by eliminating problems such as those described above. Accordingly, medical tubing bags are disclosed herein. A medical tubing bag of one embodiment includes a housing having an open interior area and generally opposed first and second ends defining respective first and second openings. A first guide is adjacent the first opening for guiding medical tubing in and out of the first opening.

In an embodiment, a medical tubing bag includes a housing having an open interior area and generally opposed first and second ends defining respective first and second openings. A first guide is adjacent the first opening for guiding medical tubing between the open interior area and an area outside the housing. The first opening has a center axis, and the first guide has a center axis; the center axis of the first opening is offset from the center axis of the first guide.

In an embodiment, a medical tubing bag for storing medical tubing that connects a user to a medical device is provided. The medical tubing bag includes a housing having an open interior area and generally opposed first and second ends defining respective first and second openings. A first guide is inwardly adjacent the first opening for guiding medical tubing into and out of the open interior area, and a second guide is inwardly adjacent the second opening for guiding medical tubing into and out of the open interior area.

In an embodiment, a medical system incorporating a medical tubing bag is provided. The medical system includes the medical tubing bag, a medical device, and medical tubing. The medical tubing bag includes a housing having an open interior area and generally opposed first and second ends defining respective first and second openings. A first guide is adjacent the first opening, and a second guide is adjacent the second opening. The medical tubing has a first end coupled to the medical device, and the medical tubing extends from the medical device through the first opening and the first guide and into the open interior area. The medical tubing continues from the open interior area through the second guide and the second opening and to a user.

### BRIEF DESCRIPTION OF THE DRAWINGS

FIG. 1 shows a side view of a medical tubing bag according to an embodiment.

FIG. 2 shows an end view of the medical tubing bag of FIG. 1.

FIG. 3 shows a sectional view of the medical tubing bag of FIG. 1.

FIG. 4 shows a guide according to an embodiment for use in the medical tubing bag of FIG. 1.

FIG. 5 shows a medical system incorporating the medical tubing bag of FIG. 1.

FIG. 6 shows a side profile of a second embodiment, and how a tubing connection is made.

FIG. 7 shows a tubing insertion process using the bag of the second embodiment.

FIG. 8 shows an embodiment having a spool tubing management system.

FIGS. 9A and 9B show a tubing management system incorporating opposing rotating members to retract or release tubing.

### DETAILED DESCRIPTION

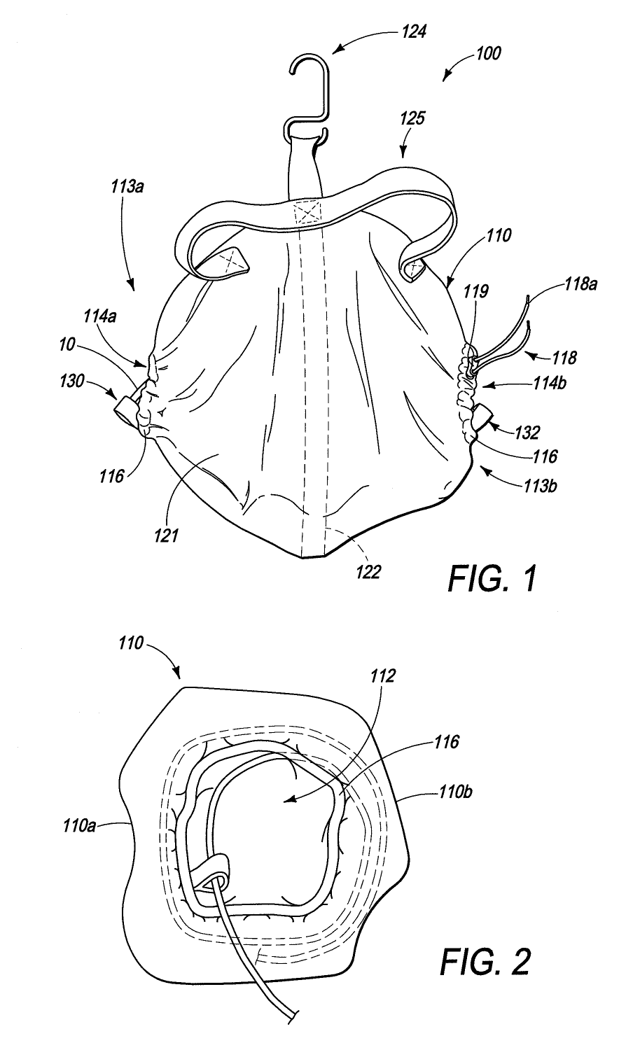

FIG. 1 through 3 show a medical tubing bag 100. The medical tubing bag 100 has a housing 110 that defines an open interior area 112. The housing 110 has generally opposed first and second ends 113a, 113b defining respective first and second openings 114a, 1 14b. The first and second openings 114a, 114b have respective perimeters that may be elastic or non-elastic. To make a respective perimeter elastic, for example, an elastic member 116 may be coupled to the housing adjacent a respective opening 114a, 114b.

The first and/or second opening 114a, 114b may be adjustable. For example, FIG. 1 shows a cord 118 that is slidably secured to the housing 110 adjacent a majority of the perimeter of the second opening 114b; a locking mechanism 119 is coupled to the cord 118 so that one or more end 118a of the cord 118 may be pulled to draw the cord 118 through the locking mechanism 119 and reduce the size of the second opening 114b. To enlarge the size of the second opening 114b, the cord 118 may be pulled through the locking mechanism 119 so that the cord ends 118a approach the locking mechanism 119. It should be appreciated that the cord 118 may be used without the locking mechanism 119 and that other adjustment devices may be used, such as a tightening strap, complementary fasteners spaced about the respective opening perimeter, etc.

The housing 110 may include a flexible fabric 121, as shown throughout the drawings, though a rigid material (e.g., plastic, wood, metal, etc.) may alternately be used. A rib 122 may be coupled to the flexible fabric 121 to maintain at least a portion of the fabric 121 at an uncollapsed configuration. In other words, the rib 122 may keep opposed sides 110a, 110b of the housing 110 separated from one another. The rib 122 may be generally centered between the first and second ends 113a, 113b of the housing 110 (FIG. 1), or the rib 122 may be offset closer to one of the ends 113a, 113b. As shown in FIG. 1, a perimeter of the fabric 121 at the rib 122 may be larger than the respective perimeters of the first and second openings 114a, 114b. The housing 110 may include an external hook 124 and/or an external handle 125 to facilitate carrying or hanging the tubing bag 100, for example.

A first guide 130 is coupled to the housing 110; the first guide 130 is adjacent the first opening 114a for guiding medical tubing 10 in and out of the first opening 114a (i.e., between the open interior area 112 and an area outside the housing 110). A second guide 132 may be coupled to the housing 110 adjacent the second opening 114b. In one embodiment, the second guide 132 may be used for guiding medical tubing 10 in and out of the second opening 114b (i.e., between the open interior area 112 and an area outside the housing 110). In another embodiment, the second guide 132 may act as a brake to generally keep an amount of medical tubing 10 from moving relative to the housing 110. For example, the second guide 132 may be sized so that a coupler connecting the medical tubing 10 with medical tubing 10 associated with a canula may not pass therethrough. If both the first and second guides 130, 132 are used to guide medical tubing 10 between the open interior area 112 and an area outside the housing 110, it may be desirable to add a braking device (e.g., a strap or loop) inside the open interior area 112 to keep an amount of medical tubing from moving relative to the housing 110.

The guides 130, 132 may be inwardly adjacent the respective openings 114a, 114b (i.e., attached to an inner surface 121a of the fabric 121 or to the elastic member 116 so as to be at least partially concealed by the housing 110, for example) or otherwise adjacent the respective openings 114a, 114b. Among other things, an inwardly adjacent configuration may be useful in guiding medical tubing 10 into the interior area 112 and in keeping the guides 130, 132 from becoming snared on various items.

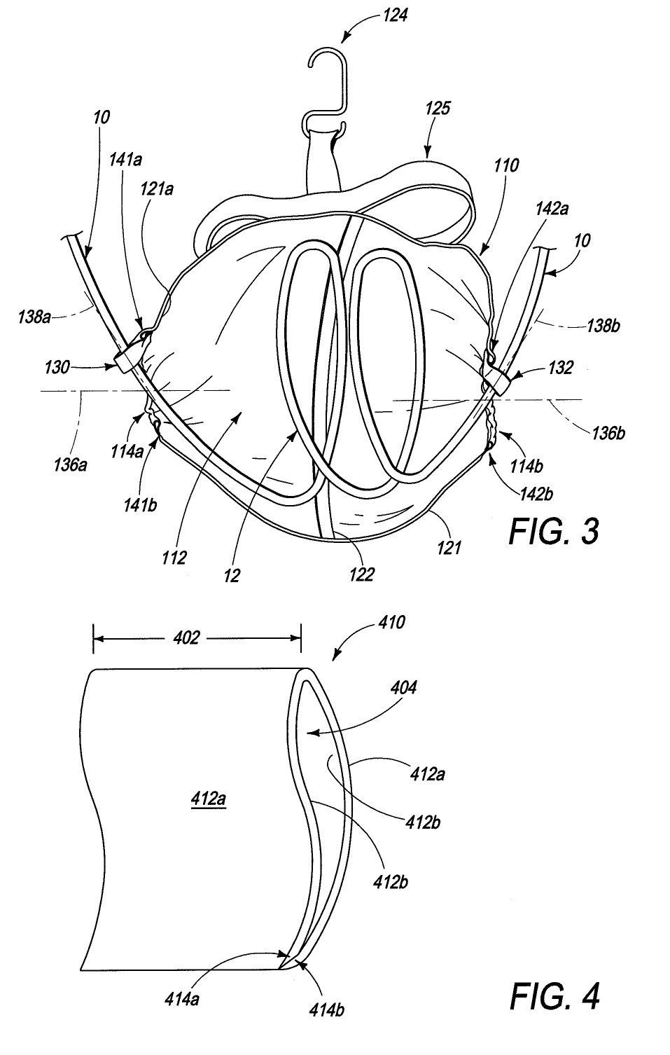

As shown in FIG. 3, the first opening 114a has a center axis 136a, the second opening 114b has a center axis 136b, the first guide 130 has a center axis 138a, and the second guide 132 has a center axis 138b. By offsetting the center axis 138a of the first guide 130 from the center axis 136a of the first opening 114a so that the axes are not parallel, the medical tubing 10 may be more easily guided in and out of the first opening 114a and the medical tubing 10 may be prompted to form a coil 12 inside the housing 110. Similarly, offsetting the center axis 138b of the second guide 132 from the center axis 136b of the second opening 114b may allow the medical tubing 10 to be more easily guided in and out of the second opening 114b, forming a coil 12 inside the housing 110. Though not specifically shown in the drawings, the first and second guides 130, 132 may be coupled to the housing 110 so that their center axes 138a, 138b are generally perpendicular with the respective center axes 136a, 136b of the openings 114a, 114b when the medical tubing 10 is separated from the guides 130, 132.

To take advantage of offsetting the first and second guides 130, 132 as described above, it may be desirable to use first and second guides 130, 132 having a significant amount of depth. For example, a guide 400 that is representative of the first guide 130 and the second guide 132 according to an embodiment is shown in FIG. 4. The guide 400 has a depth 402 and an interior diameter 404 sized to pass the medical tubing 10 therethrough. By having the depth 402 at least as large as the interior diameter 404, the offsetting may be fully taken advantage of, though other depth/diameter configurations may also be appropriate.

While various guides 130, 132 may be used, the guide 400 includes a strap 410 having inner and outer surfaces 412a, 412b and first and second ends 414a, 414b. The inner surface 412a at the first end 414a is coupled to the inner surface 412a at the second end 414b (e.g., through stitching, adhesive, etc.) to form a flexible teardrop configuration. The first and second ends 414a, 414b of the strap 410 are operatively coupled to the housing 110 (e.g., through stitching, adhesive, etc.), and as discussed above, the strap 410 may extend into the open interior area 112. In one embodiment, the strap 410 is constructed of a cloth material, though nylon and/or other appropriate materials may be used.

Returning to FIG. 3, the first opening 114a has an uppermost point 141a and a lowermost point 141b; the second opening 114b has an uppermost point 142a and a lowermost point 142b. The first guide 130 may be positioned closer to the uppermost point 141a of the first opening 114a than to the lowermost point 141b to allow the medical tubing 10 to be more easily guided in and out of the first opening 114a from a position above the housing 110. The second guide 132 may be positioned closer to the uppermost point 142a of the second opening 114b than to the lowermost point 142b to allow the medical tubing 10 to be more easily guided in and out of the second opening 114b from a position above the housing 110. This described positioning of the first and second guides 130, 132 may be desirable, for example, if the user has to reach down (e.g., to a location below the shoulder) to adjust the relationship between the medical tubing bag 100 and the medical tubing 10, which may often be necessary. If the second guide 132 is being used as a brake as mentioned above, the positioning of the second guide 132 may not be as critical as when the second guide 132 is not being used as a brake.

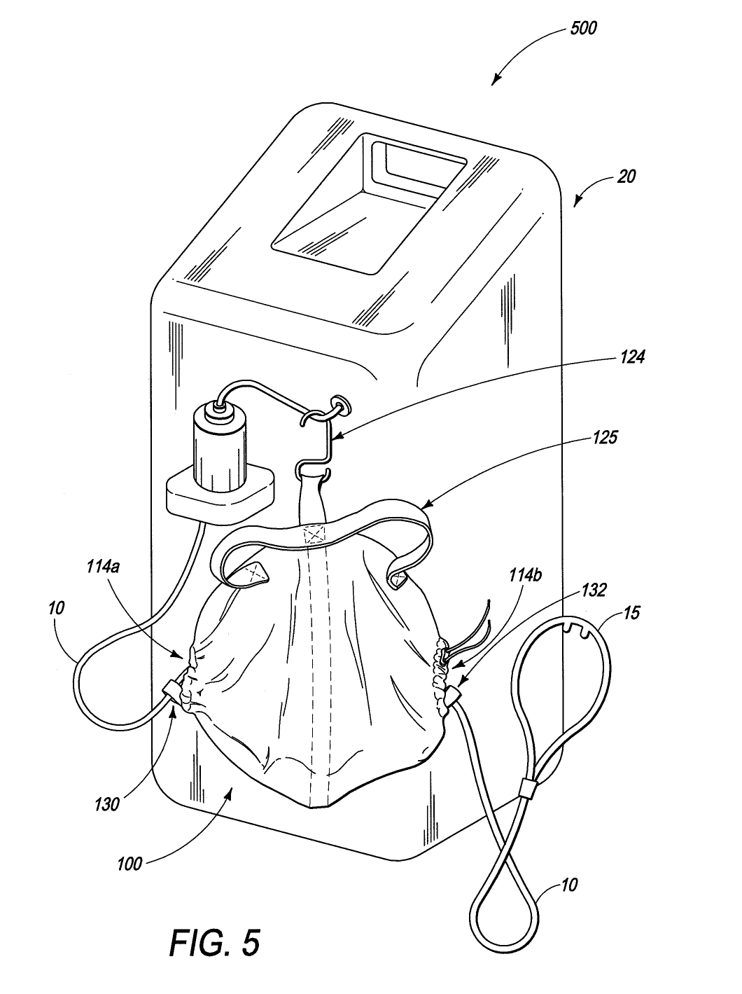

FIG. 5 shows a medical system 500 incorporating the medical tubing bag 100 described above, the medical tubing 10, and a medical device 20 (e.g., an oxygen concentrator, etc.). More particularly, a first end 10a of the medical tubing 10 is coupled to the medical device 20. The medical tubing 10 extends from the medical device 20 through the first opening 114a in the medical tubing bag 100, through the first guide 130, and into the open interior area 112. The medical tubing 10 further extends from the open interior area 112, through the second guide 132 and the second opening 114b, and to a user (e.g., through a canula 15). The amount of the medical tubing 10 in the open interior area 112 is modifiable by moving the tubing 10 through the first guide 130 and the first opening 114a and optionally by moving the tubing 10 through the second guide 132 and the second opening 114b as described above. A user may want to remove tubing 10 from the interior area 112 when moving further from the medical device 20 and introduce tubing 10 to the interior area 112 when moving closer toward the medical device 20, for example.

By forming a coil 12 inside the interior area 112 with excess medical tubing 10 as discussed above, the medical tubing 10 may be averse to kinking, easily adjusted, and generally out of the way. The first and/or second opening 114a, 114b of the medical tubing bag 100 may be adjusted as described above to allow quick access to the interior area 112 and tubing 10 housed therein. If the second guide 132 is operating as a brake as described above, it may be desirable for the first opening 114a to be adjustable, though this need not be the case. The external hook 124 may be used to attach the medical tubing bag 100 to a separate object as shown in FIG. 5 (e.g., a walker, a bed, a chair, etc.), and the external handle 125 may be used to carry the tubing bag 100.

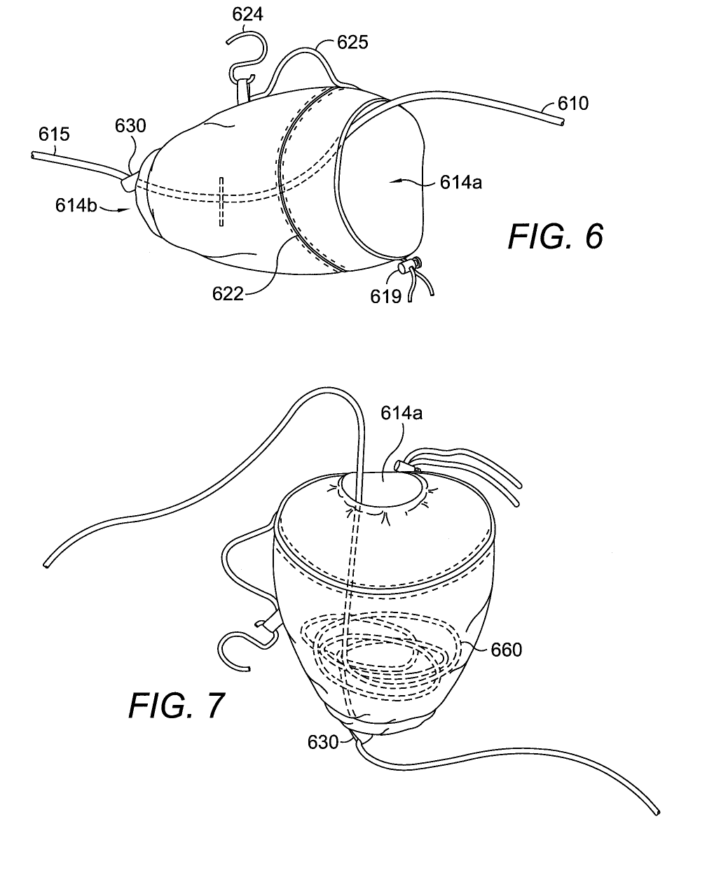

An alternative embodiment is shown in FIGS. 6-7. In this embodiment, a guide 630 is used proximate an open end 614b of the tubing extending out to the cannula. It should be noted that the concentrator tubing 610 is shown in this embodiment extending out of the cinched opening 614a of the bag and the cannula tubing 615 extends out of the other opening 614b. This is the opposite arrangement to that disclosed in FIG. 5. This arrangement shows how the bag can be used if it is carried by the patient. The patient using the cannula is thus able to move around holding the bag, while manually gathering tubing into and releasing tubing out of the cinched end.

Another difference in the FIG. 6 embodiment from the FIG. 5 version is that no guide is provided for the tubing extending out of the cinchable (using drawstring cinch 619) open end 614a going out to the concentrator. In other words, guide 130 has been removed. This, for some applications, makes it easier to gather or release the tubing.

Yet another difference from the earlier embodiments is that the outwardly compelling rib 622 in this arrangement is offset towards the side of opening 614a to a greater extent than was rib 122 in the FIGS. 1-5 embodiment. FIG. 6 shows how the tubing connection is accomplished in the bag with cinch 619 substantially loosened and rib 622 providing helpful bias. The rib positioning better distributes the outward bias proximate opening 614a where it is needed making loading easier.

It will also be observed that a handle 625 and a hook 624 have been relocated in this embodiment which makes it more easily used in some applications.

FIG. 7 shows how the concentrator tubing can be inserted/returned into the bag to create a looped bundle 660 in the interior of the bag. During tubing insertion, the drawstring is pulled to significantly reduce the size of opening 614a to prevent loops of tubing from escaping the bag. Guide 630 functions in much the same ways discussed for the guides in the earlier embodiments.

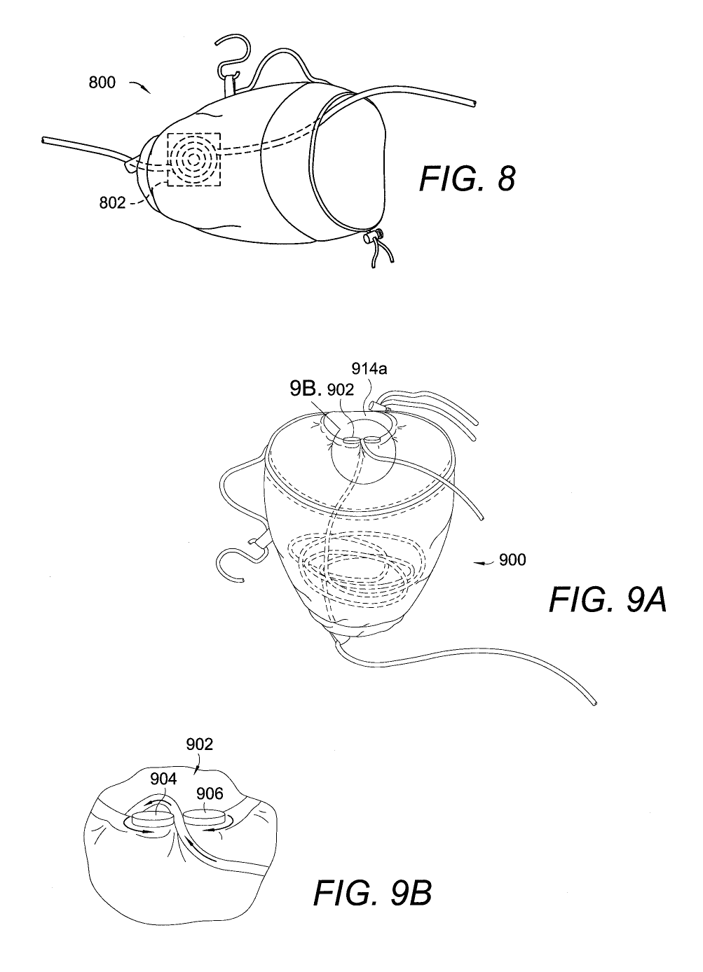

Yet another embodiment is disclosed in FIG. 8. The FIG. 8 embodiment is substantially the same as the one shown in FIGS. 6-7, except that a tubing handling system 802 is provided in the bag. In this embodiment, handling system 802 includes an electrically-driven spooling arrangement. Those in the art will recognize that these sorts of devices are activated by a user, e.g., using a switch, to receive the tubing onto a rotating receiving member (e.g., a spool). In addition to being able to retract, reverse rotation will unspool, thus release the tubing. Reel 802 in some embodiments is sewn, adhered, or otherwise fixed into the bag interior. In other embodiments, it could be loosely held. As will be apparent to those in the art, reel 802 automates the processes of receiving the tubing into the bag, as well as releasing if desired. Another variable which should be understood is that while in one embodiment provides the spooling arrangement is adapted to receive or release the tubing leading to the cannula, another embodiment would involve the retraction or release of the tubing leading to the concentrator.

An alternative tubing handling system 902 is disclosed in FIGS. 9A and 9B. This kind of system provides a pair of opposed rotating members 904 and 906 which can be seen in detail in FIG. 9B. The opposing edges of members 904 and 906 include annular channels which together match the shape of, receive and engage the outside surfaces of the tubing from both sides. When members 904 and 906 are counter rotated relative to one another in a first direction (e.g., that shown in FIG. 9B) the tubing will be directed into the bag as shown in FIG. 9A. When members 904 and 906 are counter rotated in a second opposite direction, the tubing will be drawn out of the bag (this is not shown). By making these directions of rotation controllable by the user, that user is able to either retract or release the tubing. Like with the FIG. 8 arrangement, the reel of FIGS. 9A and 9B could be adapted to receive or release the tubing leading to the cannula, or alternatively, retract and release the tubing leading to the concentrator.

Those skilled in the art appreciate that variations from the specified embodiments disclosed above are contemplated herein. The description should not be restricted to the above embodiments, but should be measured by the following claims.

What is claimed is:

1. A medical tubing bag comprising:
an open interior area and an opening, said opening including a mechanism for varying a size of said opening; and
a tubing handling mechanism for one of retracting and expelling said tubing from said opening.
